(19) United States
(12) Patent Application Publication
(10) Pub. No.: US 2017/0181795 A1
(43) Pub. Date: Jun. 29, 2017

(54) LIMITED ABLATION FOR THE TREATMENT OF SICK SINUS SYNDROME AND OTHER INAPPROPRIATE SINUS BRADYCARDIAS

(71) Applicant: DR PHILIPPE DEBRUYNE BVBA, Tervuren (BE)

(72) Inventor: Philippe Debruyne, Tervuren (BE)

(21) Appl. No.: 15/450,467

(22) Filed: Mar. 6, 2017

Related U.S. Application Data
(63) Continuation-in-part of application No. PCT/EP2015/073634, filed on Oct. 13, 2015.

(30) Foreign Application Priority Data
Oct. 13, 2014 (EP) PCT/EP2014/071914

(51) Int. Cl. A61B 18/14; A61B 5/06; A61B 6/00; A61B 18/24

(52) U.S. Cl. CPC A61B 18/1492; A61B 18/24; A61B 5/066; A61B 6/503; A61B 2018/00357

(57) ABSTRACT

The current invention concerns a method of ablation designed for the treatment of sick sinus syndrome and other medical conditions characterized by abnormal sinus bradycardia. The method includes the steps of inserting an ablation catheter into a heart of a living subject and directing energy from the ablation catheter towards tissue at a targeted location for ablation. In the method, a specific limited location at level of the junction between the right atrium and the superior vena cava is targeted.

### TECHNICAL FIELD

The invention pertains to the technical field of minimally invasive treatments of organs inside the body of a living subject. More specifically, this invention pertains to a method and system for the treatment of a cardiac arrhythmia.

### BACKGROUND

Tachyarrhythmias and ectopic heart rhythms can be treated by selectively ablating cardiac tissue by application of energy via a catheter. Bradyarrhythmias are usually treated by pacemaker implantation.

The rhythmic activity of the heart is due to the spontaneous diastolic depolarization of specialized cells located subepicardially near the lateral right side of the junction between the superior vena cava and the right atrium and forming the sino atrial node or sinus node. A dysfunction of the sinus node or a sick sinus syndrome is a frequent cardiac disorder, which can lead to exercise limitation, to dizziness and even to syncope. When the sinus node dysfunction is clinically relevant, a pacemaker is commonly recommended, usually involving a dual chamber pacemaker. Pacemaker implantation is commonly performed but comprises still potential risks (pericarditis, cardiac tamponade, pocket infections, endocarditis, pneumothorax, subclavian occlusion, diaphragm stimulation, death, etc.). Depending on the pacemaker recipient, several generators replacements, electrode extractions and replacements can also be needed, leading to additional risks. When chronotropic incompetence is clinically relevant, heart rate acceleration can be determined by activation of different sensors in the device but the kinetics of this heart rate acceleration is very often suboptimal compared to those achieved by a healthy sinus node during exercise. Esthetical problems and life style limitations can also be problematic in young patients.

Taking these problems and potential risks of pacemaker implantation into mind, alternatives to this commonly used method should be considered. Administration of medication is not an option, since no oral drugs are currently available to improve sinus node function.

There remains a need in the art for alternative treatments for sick sinus syndrome and other medical conditions characterized by an abnormal functional bradycardia (cardio-inhibitory syncope, hypersensitivity of the carotis sinus), effectively leading to an enhancement of the sinus node function in those patients, and consequently avoiding the placement of a pacemaker.

### SUMMARY OF THE INVENTION

An embodiment of the invention provides a method for ablation, designed for the treatment of sick sinus syndrome and other medical conditions characterized by an abnormal functional bradycardia, which is carried out by inserting an ablation catheter into a heart of a living subject and directing energy from the ablation catheter towards tissue at a targeted location for ablation thereof. In the method, the targeted location corresponds to a specific limited location at level of the junction between the right atrium and the superior vena cava. In an aspect of the method, the specific limited location is targeted from the endocardial side of the right atrium. In another aspect of the method, the specific limited location corresponds to a definite small region of a few millimeters at the posterior side of the junction between the superior vena cava and the right atrium and opposed to the junction of the right superior pulmonary vein with the left atrium. Another aspect specifies that the right anterior ganglionated plexi are targeted for ablation at the specific location. In another aspect of the method, it is provided that sinus node acceleration is obtained during the ablation treatment. Yet another aspect of the method provides that the amount of energy applied for ablation and the surface of the ablation area are determined by evaluating sinus rhythm acceleration.

Another embodiment of the invention provides a system for carrying out the method described above.

A final embodiment of the present invention comprises a manual for carrying out the method described above.

### DESCRIPTION OF FIGURES

For a better understanding of the present invention, reference is made to the detailed description of the invention, by way of example, which is to be read in conjunction with the following drawings, wherein:

FIG. 1 is a diagram of the right atrium, left atrium, sinus node, caval and pulmonary veins of a heart in the posteroanterior view depicting the ablation target in accordance with an embodiment of the present invention;

FIG. 2 is a diagram of a possible design of an ablation catheter in accordance with an embodiment of the present invention.

FIG. 3 shows a three-dimensional representation of an ablation catheter in accordance with an embodiment of the present invention.

FIG. 4 is a diagram of the right atrium, left atrium, sinus node, caval and pulmonary veins of a heart in the posteroanterior view depicting a specific limited location for ablation in accordance with an embodiment of the present invention.

FIG. 5 is a diagram of the right atrium, left atrium, sinus node, caval and pulmonary veins of a heart depicting a specific limited location for ablation in accordance with an embodiment of the present invention. FIG. 5A shows a posteroanterior (PA) view. FIG. 5B shows a left anterior oblique (LAO) view. FIG. 5C shows an anterior posterior (AP) view.

FIG. 6 is an outline of a time course of a living subject's heart rate during and after ablation, according to embodiments of the present invention.

FIG. 7 is a graph showing P-P interval shortening as a function of time during two applications of radiofrequency ablation at a specific limited location, according to embodiments of the present invention.

FIG. 8 is a graph showing the residual amount of the P-P interval value retained during follow up after ablation treatment at a specific limited location according to an embodiment of the present invention.

FIG. 9 is a graph showing the residual amount of the P-P interval value during follow up after ablation treatment at a specific limited location according to an embodiment of the present invention.

FIG. 10 is a schematical representation of an algorithm for ablation according to embodiments of the present invention.

FIG. 11 is a graph showing the periprocedural HR modifications as tracked by a non invasive HR monitoring, before, during and after ablation at a specific limited location according to an embodiment of the present invention.

FIG. 12 shows P-P interval results as monitored before and after ablation at a specific limited location according to an embodiment of the present invention.

FIG. 13 shows heart monitoring results as monitored before and after ablation treatment at a specific limited location according to an embodiment of the present invention.

FIG. 14A-B are diagrams showing (14A) a left atrium and pulmonary veins of a heart in an anterior posterior view and (14B) a left atrium, right atrium, pulmonary veins and caval veins of a heart in posteroanterior view, with indication of landmark lines for ablation, in accordance with an embodiment of the present invention.

FIG. 15A-E show diagrams related to different steps intended for indicating landmark lines for ablation on a heart and for performing ablation at level of one of said lines, in accordance with an embodiment of the present invention. FIG. 15A shows a first landmark line 17 indicated on a CT image on a junction between the left atrium 2 and a right superior pulmonary vein 6 of an anterior posterior view as obtained by a CT scan of said heart. FIG. 15B shows a posteroanterior view of a CT image of said heart in a second landmark line 20 indicated at a junction between a superior vena cava 5 and a right atrium 3 of said heart by performing a perpendicular projection of said first landmark line 17 onto said junction between a superior vena cava 5 and a right atrium 3 of said heart. FIG. 15c shows right cardial structures, among which the right atrium 3, superior vena cava 5, inferior vena cava 4 and coronary sinus 16, are mapped, after which the resulting map is merged with the original CT image. The thus resulting image of heart structures is shown according to a left anterior oblique view. FIG. 15D shows ablation performed at such point location 23, as shown in a posteroanterior view. FIG. 15E shows in an anterior posterior view that, said point location 23 is also located along said first landmark line 17.

### DETAILED DESCRIPTION OF THE INVENTION

Unless otherwise defined, all terms used in disclosing the invention, including technical and scientific terms, have the meaning as commonly understood by one of ordinary skill in the art to which this invention belongs.

“A”, “an”, and “the” as used herein refers to both singular and plural referents unless the context clearly dictates otherwise. By way of example, “a compartment” refers to one or more than one compartment.

“Comprise”, “comprising”,and“comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

In the following description of the invention, numerous specific details are set forth in order to provide a thorough understanding of the various principles of the present invention. It will be apparent to one skilled in the art, however, that not all these details are necessarily always needed for practicing the present invention.

A new opinion concerning the underlying cause of some sinus bradycardias constitutes the starting point for this invention. In scientific literature, sick sinus syndrome is commonly described as a degenerative disease, correlated with fibrosis in the atrium. Many episodes of bradycardias are however intermittent, which is not consistent with the notion of sick sinus syndrome as a degenerative disease. Therefore, it is proposed that at least a subgroup of patients with symptomatic bradycardias rather exhibit an inadequate balance of the cardiac autonomous nervous system. Those patients could be selected based on the heart rate acceleration provided by intravenously injection of a vagolytic agent (for example, atropine). In this context, the importance of ganglionated plexi surrounding the atria of the heart in the genesis of atrial fibrillation has been extensively investigated during the last decennia and has been proposed as a target to treat atrial fibrillation. The sinus node is innervated by the anterior right ganglionated plexi.

The present invention aspires to treat a subgroup of patients with sick sinus syndrome reversible by vagolytic treatment through a limited endocardial ablation at a specific location at level of the junction between the right atrium and the superior vena cava, intended to ablate the anterior right ganglionated plexi. It is presumed that the same ablation approach can be used for the treatment of other inappropriate sinus bradycardias than sick sinus syndrome which are reversible by vagolytic treatment.

In a first embodiment, the invention provides a method of ablation for the treatment of sick sinus syndrome and other medical conditions characterized by abnormal sinus bradycardia, comprising the steps of: inserting an ablation catheter into a heart of a living subject, and directing energy from the ablation catheter towards tissue at a targeted location for ablation thereof, wherein the targeted location corresponds to a specific limited location at level of the junction between the right atrium and the superior vena cava.

From the above, it is clear that the specific limited location is a specific epicardial structure. In preferred embodiments, the site for ablation, being the specific limited location, is approached endocardially.

In a preferred embodiment, the specific limited location is situated in front of the junction between the right atrium and the superior vena cava, and in particular in front of the inferior and mid parts of the septal aspect of the right superior pulmonary vein.

In a preferred embodiment, the specific limited location is situated at level of the junction between the right atrium and the superior vena cava, yet rather on the side of the superior vena cava.

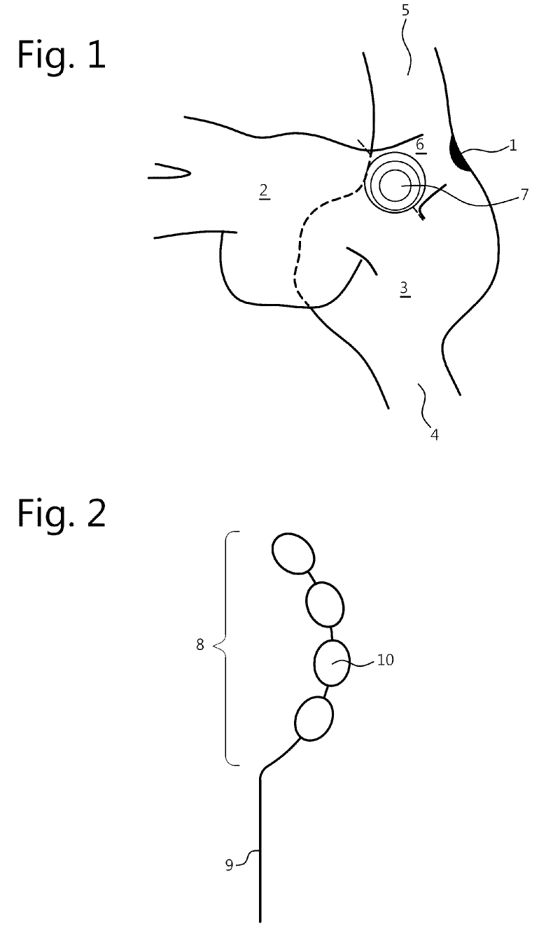

For the specific ablation target 7, according to the method of the present invention, reference is made to FIG. 1, showing the right atrium 3, left atrium 2, sinus node 1, caval and pulmonary veins of a heart in the posteroanterior view. The ablation target 7 is located between the right 3 and the left atrium 2 and their respective venous connections. According to a preferred embodiment of the invention, the ablation target 7 is targeted from the endocardial side of the right atrium 3. Exceptionally, a left atrial side approach or a pericardial approach could be proposed based on the anatomical characteristics of the patient to treat. For safety reasons, those alternative approaches will not be privileged. To approach the ablation target 7 from the endocardial side of the right atrium 3, an ablation catheter is introduced in the right atrium 3. The ablation target 7 corresponds to a definite small region of a few millimeters at the posterior side of the junction between the superior vena cava 5 and the right atrium 3 and opposed to the junction of the right superior pulmonary vein 6 with the left atrium 2. Furthermore, the ablation target 7 is located in front of the superior and anterior part of the right antrum, indicated by a dotted line in FIG. 1. Based on the relationship between the left 2 and right atrium 3, the definite small region is sometimes located more septal or more cranial, which is indicated by circles around the ablation target 7 on FIG. 1. In a preferred embodiment of the present invention, the specific limited location of the ablation target 7 ranges from 5 to 10 millimeters in diameter. The small region of the ablation target 7 can be easily located after preparing a detailed anatomical map of the right atrium 3 and integration of this map (“merge”) with a previous anatomical delineation of both atria (for example by a CT scan), like shown on FIG. 1. At the ablation target 7, the anterior right ganglionated plexi are targeted for ablation. This ablation of the anterior right ganglionated plexi at the ablation target 7 is intended to obtain an enhancement of the sinus node 1 function by neuromodulation.

In a preferred embodiment, the small region of the ablation target 7 can be easily located after preparing a detailed anatomical map of the right atrium 3 and integration of this map with a previous anatomical delineation of both the atria and the pulmonary veins (for example by a CT scan).

In embodiments, the specific limited location 7 or ablation target 7 is targeted from the endocardial side of the right atrium 3 or superior vena cava 5, and in particular from the junction between the right atrium 3 and the superior vena cava 5, either on atrial side or on venous side.

During the ablation treatment, sinus node acceleration is obtained. In particular, the ablation of the anterior right ganglionated plexi, which diminishes or annihilates the vagal innervation of the sinus node, brings about heart rate acceleration. The energy applied for ablation and the surface of the ablation area are determined by evaluating the heart rate acceleration or, in other words, sinus rhythm acceleration.

In an embodiment, sinus rhythm acceleration is evaluated by evaluating P-P interval shortening.

The response in heart rate can be titrated according to the energy transfer to the target. The heart rate typically accelerates progressively according to the ablation time. The heart rate targeted during ablation must be higher than the desired basal heart rate after treatment. The required amount of energy for ablation corresponds to the amount of energy necessary to reach this targeted heart rate or, in other words, to reach a predefined amount of sinus node acceleration. This predefined amount of sinus node acceleration can be utilized for automatic regulation of ablation energy delivery. A program or device responsible for the delivery of ablation energy can be configured to apply energy for ablation according to the extent in which the predefined amount of sinus node acceleration is reached. When this predefined amount of sinus node acceleration is reached, the supply of energy will be automatically terminated. It must be emphasized that the energy suitable for the ablation treatment, according to the method of the present invention, is not restricted to a particular type of energy. As well radiofrequency energy, laser energy, microwave energy, cryogenic cooling, as ultrasound energy can serve as ablation energy in the method of the present invention. Although an ablative treatment, utilizing a catheter, is put forward in embodiments of the present invention for directing energy towards the right anterior ganglionated plexi at the specific limited location 7, this location 7 could also be targeted using radiotherapy. A focal lesion could be created at level of the specific limited location 7 using radiotherapy. Proton therapy is a type of radiotherapy which could be used for this application.

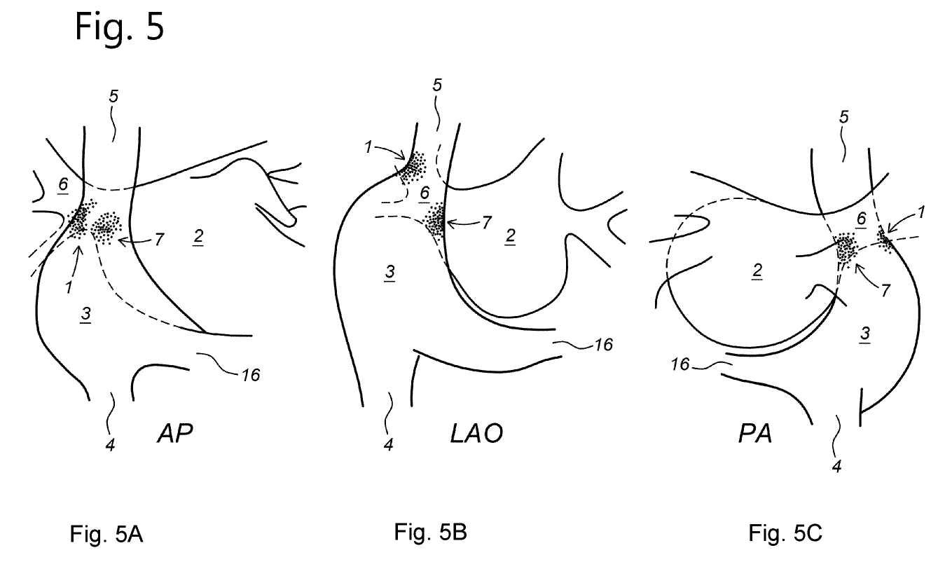

In a preferred embodiment, the present invention provides a method according to the method of the invention, whereby the specific limited location 7 corresponds to a definite small region of a few millimeters at the posterior side of the junction between the superior vena cava 5 and the right atrium 3 and opposed to the junction of the right superior pulmonary vein 6 with the left atrium 2, at level of the inferior and mid parts of the right superior pulmonary vein 6. Sinus node acceleration is most efficient at this location 7. FIG. 4 shows a diagram of a part of a heart in the posteroanterior view depicting the specific limited location 7 for ablation in accordance with this embodiment. FIG. 5 shows diagrams of a part of a heart in the posteroanterior (PA), left anterior oblique (LAO) and anterior posterior (AP) view depicting a specific limited location for ablation in accordance with this embodiment.

In preferred embodiments, the method of ablation at the specific limited location 7 according to the present invention is intended as a treatment to increase sinus node basal activity, to avoid pathological pauses and/or to perform non pharmacological vagolysis of the sinus node.

In a preferred embodiment, the present invention provides a method according to the method of the invention, whereby the specific limited location 7 ranges from 3 to 17 millimeters in diameter, and more preferably from 5 to 15 millimeters in diameter.

In a preferred embodiment, the present invention provides a method according to the method of the invention, whereby the specific limited location 7 ranges from 10 to 20 millimeters in diameter, and more preferably from 10 to 15 millimeters in diameter.

In a preferred embodiment, the present invention provides a method according to the method of the invention, whereby the method further comprises a screening step in which patients are screened prior to ablation and a follow up step in which patients are followed up after ablation at the specific limited location 7. In particular for the screening step, the patients are screened if the method of ablation would be beneficial for them. As mentioned above, patients which are suitable for the method of ablation according to the invention are selected based on the heart rate acceleration provided by intravenously injection of a vagolytic agent, such as atropine. In embodiments, patients are screened by pharmacological vagolysis using atropine. Preferably, the patients should be very relaxed. Patients with significant infra-nodal conduction disturbances are not fit for the method of ablation according to the invention. The P-P interval may be monitored by any known portable ECG monitoring device.

In an embodiment of the follow up step, it is monitored to which extent the heart rate increases and P-P interval decreases as affected by the ablation treatment are maintained. In particular, the heart rate and/or P-P interval of the patients are monitored, in order to follow up these parameters after an ablation treatment.

In a preferred embodiment, the living subject, of which the heart will be subject to an ablation at the specific limited location 7 according to the method of the present invention, is anesthetized prior to ablation. This is advantageous, since patients which would undergo the procedure under conscious sedation could have a higher catecholaminergic status, which could lead to the interpretation of heart rate modifications as a response to pain.

In order to determine the specific limited location 7 precisely, it is mandatory to know the locations of both left and right atrio-venous structures. Therefore, in embodiments, the locations of both left and right atrio-venous structures are mapped. For this purpose, the right atrium 3, superior vena cava 5, right superior pulmonary vein 6, inferior vena cava 4 and/or coronary sinus 16 are visualized in embodiments.

In a preferred embodiment, the present invention provides a method according to the method of the invention, whereby the specific limited location 7 is identified by imaging both the left and right atrio-venous structures. The term “imaging”, as used herein, can, among others, refer to electroanatomical mapping, CT scans, the merging of electroanatomical maps with CT scans, and filming the venous return of one or more venous structures injected with contrast fluid.

In a preferred embodiment, the present invention provides a method according to the method of the invention, whereby the specific limited location 7 for ablation is mapped by introducing a diagnostic catheter in the coronary sinus 16 and subsequently constructing an electroanatomical map of the right atrium 3, the caval veins and the proximal part of the coronary sinus 16. These electroanatomical maps are subsequently merged with CT scans of the right atrium 3 and the left atrium 2.

In a preferred embodiment, the present invention provides a method according to the method of the invention, whereby the specific limited location 7 for ablation is mapped by introducing a diagnostic catheter in the coronary sinus 16, followed by constructing an electroanatomical map of the right atrium 3, the caval veins and the proximal part of the coronary sinus 16, and also by delayed imaging of the right superior pulmonary vein 6 after selective angiography of the right superior pulmonary artery. Said delayed imaging of the right superior pulmonary vein 6 after selective angiography of the right superior pulmonary artery is preferably assisted by a high flow pomp and also preferably assisted by a pigtail catheter. The imaging is preferably performed by filming the venous phase after a contrast injection in the right superior pulmonary artery. This can be performed by injecting approximately 45 mL of contrast fluid with a flow rate of 15 mL/s, and filming between 5 s and 10 s after the start of the injection in a left anterior oblique view position adapted to each patient. This embodiment avoids a left atrial side approach or the potential errors related to a manual merge when using a CT scan, which leads to an improved safety of the method. Furthermore, the approach of this embodiment will diminish patient irradiation and will save time and energy consumption for both patients and the community. In embodiments, information obtained by the last mentioned approach is incorporated in a navigation system enabled to fuse electro-anatomical maps with procedural X-Rays pictures, such as, for example, the CartoUnivu™ technology of Biosense Webster, Diamond Bar, Calif., USA. Injecting amounts of contrast fluid lower than the approximately 45 mL is also possible. In patients with kidney function impairment, the volume of contrast fluid injected can be limited by a selective injection within the superior branch of the right pulmonary artery.

In embodiments, the right superior pulmonary vein 6 is imaged by constructing an electroanatomical map of the left and right atrio-venous structures. In other embodiments, the right superior pulmonary vein 6 is imaged by performing CT scans of both atria and subsequently merging the CT scans. In still other embodiments, the right superior pulmonary vein 6 is imaged using intracardiac echocardiography.

In a preferred embodiment, the present invention provides a method according to the method of the invention, whereby the specific limited location 7 for ablation is mapped by introducing a diagnostic catheter in the coronary sinus 16, followed by constructing an electroanatomical map of the right atrium 3, the caval veins and the proximal part of the coronary sinus 16, and also by visualization of the right superior pulmonary vein 6 by intra-cardiac ultrasounds. The use of ultrasounds is a highly non-destructive visualization technique.

In a preferred embodiment, the present invention provides a method according to the method of the invention, whereby the specific limited location 7 for ablation is mapped by introducing a diagnostic catheter in the coronary sinus 16, followed by maping of right atrium 3, left atrium 2 and the caval veins and the proximal part of the coronary sinus 16, and also by endocardial mapping of the right superior pulmonary vein 6. This embodiment is especially suitable for an ablation treatment which combines ablation at the specific limited location 7 with pulmonary vein ablation.

In a preferred embodiment, the present invention provides a method according to the method of the invention, whereby the ablation catheter comprises multiple electrodes for performing ablation, and whereby at least one of the multiple electrodes, yet preferably 4 to 5 electrodes, direct energy towards tissue at the specific limited location 7 for ablation thereof. The simultaneous targeting of limited locations within the specific limited location 7 is advantageous since it diminishes edema formation within the location 7, when compared to separate targeting of such limited locations.

In a preferred embodiment, the present invention provides a method according to the method of the invention, whereby the ablation catheter delivers 5 W to 100 W, more preferably 10 W to 80 W, even more preferable 15 W to 70 W, and most preferably 20 W to 55 W of energy to the specific limited location 7.

In a preferred embodiment, the present invention provides a method according to the method of the invention, whereby the ablation catheter delivers at least once energy to the specific limited location 7 during a treatment time of 5 s to 200 s, more preferably during 10 s to 150 s, even more preferably during 20 s to 120 s, yet even more preferably during 30 s to 90 s, and most preferably during 45 s to 75 s. In a most preferred embodiment, the ablation catheter delivers at least once ablation energy to the specific limited location 7 during a treatment time of 60 s. The energy delivery of the catheter to the specific limited location 7 during abovementioned treatment times can be repeated a number of times. In embodiments, the energy delivery according to abovementioned treatment times is performed 2 to 15 times.

Performing the ablation with abovementioned energy values and during abovementioned treatment times warrants sufficient ablation at the specific limited location 7 while avoiding undesired edema formation at level of the location 7.

In a preferred embodiment, the present invention provides a method according to the method of the invention, whereby the ablation catheter is irrigated while the ablation catheter is directing energy towards tissue at the specific limited location 7. Irrigation of the ablation catheter serves to reduce excessive heating of tissue and blood at the specific limited location 7, preventing the occurrence of thrombus and char formation and thus enabling the creation of larger lesions. In a preferred embodiment, the ablation catheter is irrigated by circulating a suitable fluid, such as a saline fluid, through or around the ablation catheter with an irrigation flow rate of 1 mL/min to 60 mL/min, more preferably of 2 mL/min to 50 mL/min, even more preferably of 5 mL/min to 40 mL/min, and most preferably of 10 mL/min to 35 mL/min.

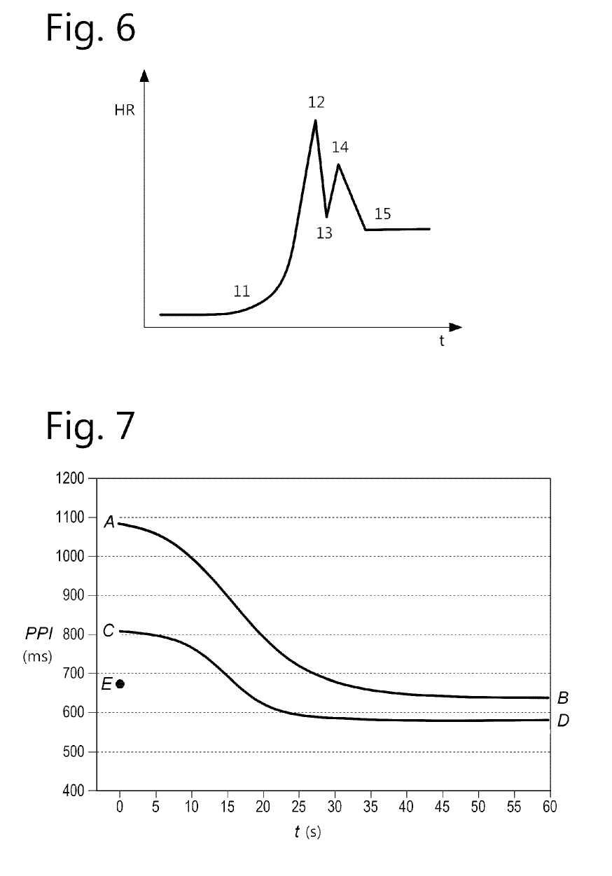

As mentioned above, the heart rate targeted during ablation must be higher than the desired basal heart rate after treatment. This is important because a part of the heart rate acceleration achieved by ablation is lost afterwards. An outline of a time (t) course of a living subject's heart rate (HR) during and after ablation, according to embodiments of the present invention, is depicted in FIG. 6. Prior to treatment, the heart rate equals an initial basal heart rate 11. Due to the ablation of the specific limited location 7, an acute increase to a higher heart rate level 12 is increased. This higher heart rate level 12 is the heart rate targeted during ablation 12. Part of said increase in heart rate is permanent while another part is lost, resulting in a post ablation heart rate 13. This post ablation heart rate 13 will subsequently increase to a higher level 14 due to the cessation of anesthesia. Ultimately, the heart rate develops into a post procedural heart rate 15 which is higher than the initial basal heart rate 11. Furthermore, the post procedural heart rate's 15 decrease in time is limited. In a preferred embodiment, the post procedural heart rate 15 at 4 months after the ablation is at least 80%, more preferably at least 85% and most preferably at least 90%, of the heart rate targeted during ablation 12.

Next to an increase in heart rate, ablation also leads to a reduction of the P-P interval. A P-P interval is commonly known as the distance between consecutive P waves in an electrocardiogram. In embodiments, the P-P interval as determined directly after ablation at the specific limited location 7 is at most 75%, preferably at most 70%, and more preferably at most 65% of the P-P interval prior to ablation. In embodiments, the P-P interval determined 6 months after ablation at the specific limited location is at most 80%, preferably at most 75% and more preferably at most 70% of the P-P interval prior to ablation. The ablation treatment according to the method of the present invention thus leads to a persistent biological effect.

In a preferred embodiment, the present invention provides a method according to the method of the invention, whereby at least identification of the specific limited location 7, imaging of the right superior pulmonary vein 6, ablation at the specific limited location 7, screening of the patients prior to ablation, and/or follow up of patients after ablation, is regulated by an algorithm.

In an embodiment, an algorithm is provided which is intended to regulate the identification of the specific limited location 7.

In an embodiment, an algorithm is provided which is intended to regulate the imaging of the right superior pulmonary vein 6. Such an algorithm will regulate the amount of contrast fluid, the kinetics of contrast fluid injection, and the latency of filming the venous return, by evaluating basal parameters and procedural parameters. Said basal parameters are dependent on the patient and include, among others, heart rate and invasive pressure. Said procedural parameters include, among others, the position of a diagnostic and preferably pigtail catheter for the imaging. During imaging, the diagnostic catheter may be present in, among others, the right ventricle, the pulmonary trunk, the right pulmonary artery or the superior part of the right pulmonary artery.

In an embodiment, an algorithm is provided which is intended to regulate the screening of patients prior to ablation. In such an algorithm, a basal P-P interval is determined by determination of the mean value of consecutive P-P intervals, such as, for example, 6 consecutive P waves without supraventricular extrasystoles. Subsequently, a pharmacological test with increasing doses of atropine is performed. The mean P-P interval post atropine should be short enough, such as, for example, less than 900 ms, and the P-P interval shortening should be significant, such as, for example, at least 20%.

In an embodiment, an algorithm is provided which is intended to regulate the follow up of patients after ablation at the specific limited location 7. In an embodiment of such algorithm, average P-P intervals are registered on basis of multiple consecutive P waves at rest, preferably 6 consecutive P waves at rest, thus under same basal conditions. In an embodiment, said P waves at rest are registered with a compact ECG registration device with or without external electrode cables. An Omron® Portable HeartScan ECG Monitor may be used for this purpose. In an embodiment, the follow up of patients is executed at home.

Following up patients at their home environment is beneficial for their well-being, since the transportation to a hospital, clinic or private clinical practice for at least part of follow up is made unnecessary. In embodiments, patients are provided with electrodes which can be connected to a mobile device, such as a mobile phone, tablet or portable computer, and/or to a non-mobile device such as a non-portable computer. Preferably, the patients are provided with electrodes connected to a mobile device. A specific program for managing and storing measured data could be delivered. For example, such program could be offered as a downloadable program. The practicing physician can subsequently analyze the stored data at a later stage. In an embodiment, the measured data could be stored in a large central database, which is preferably an anonymous database.

In an embodiment, an algorithm is provided which is intended to regulate ablation at the specific limited location. Such embodiment is useful to improve catheter localization and determination of ablation pre-settings, and said algorithm enables to adapt ablation parameters dynamically during ablation. In embodiments, said algorithm incorporates basal parameters, both anatomical and functional, a pre-specified desired biological effect, for example, the desired basal heart rate after ablation, and life procedural data. The algorithm preferably a feed-back mechanism which will show ideal catheter positioning during ablation and which will continuously adapt ablation parameters during ablation, define number of applications and their duration. Such algorithm is highly desired since it can be used to tailor an ablation treatment to patient's needs.

In embodiments, said algorithm is proposed on observed time-effect typical sigmoid curves of P-P interval while ablating on adequate ablation sites, and on the partial “vanishing effect” after each application. This “vanishing effect” is used in this text to denominate the post procedural increase in P-P interval after the ablation. The post procedural P-P interval of a patient is however lower than the P-P interval of that patient prior to ablation. In other words, the ablation leads to a persistent biological effect. Following the proposing of the algorithm, an operator must choose a desired basal heart rate to be achieved directly after ablation treatment. Subsequently, the algorithm provides the location where the first application of energy, such as, for example, radiofrequency energy, should be delivered, next to ablation parameters such as, for example, the amount of required energy expressed in Watts. Subsequently, the algorithm evaluates the effect of the ablation treatment and informs the operator if the contact with the endocardium must be enhanced or if a catheter replacement or displacement is needed. Typically, the biological response is evaluated between 15 seconds and 20 seconds after the start of ablation, after which the response is continuously tracked with online creation of time-response curves. Additionally, the algorithm is able to indicate how many applications are needed to achieve a particular heart rate at follow up.

Although the present invention provides a method of ablation for the treatment of sick sinus syndrome and other medical conditions characterized by abnormal sinus bradycardia, the proposed method may well be used for the treatment of other cardiac disorders. For example, the method of ablation according to the present invention may well be of some utility for patients with atrial fibrillation or for patients with long QT syndrome.

In a preferred embodiment, the present invention provides a method according to the method of the invention, wherein before the step of directing energy from the ablation catheter towards tissue at a targeted location for ablation thereof, the method comprises the step of acquiring an assembly of one or more images and/or maps which as a whole at least show the junction between the heart's left atrium and right superior pulmonary vein, as well as the heart's right atrium and superior vena cava, and subsequently the step of indicating a first landmark on the junction between the left atrium and right superior pulmonary vein on one or more images and/or maps, and the step of indicating a second landmark, which second landmark comprises at least one targeted location corresponding to a specific limited location, by performing a perpendicular projection from said first landmark on the junction between the right atrium and the superior vena cava on one or more images and/or maps.

When visually perceived on one or more images and/or maps, which may be merged for visualization goals, said first and second landmarks may be observed as being located behind each other. Said assembly of one or more images and/or maps may comprise exactly one image or map, or may comprise any number of images and/or maps. Said images and/or maps may be acquired by any imaging and/or image processing tools as known in the state of the art. In an embodiment, images and/or maps are acquired by applying one or more computer tomography (CT) scans. In other embodiments, three-dimensional image data are recorded by use of a X-ray CT scan and/or magnetic resonance tomography. In still other embodiments, images and/or maps are acquired by right pulmonary angiography or by taking ultrasound images. In a preferred embodiment, right pulmonary angiography is performed and in the levophase of an image resulting from said right pulmonary angiography said first landmark is indicated on the junction between the left atrium 2 and the right superior pulmonary vein 6, after which this first landmark is directly projected on the junction between the right atrium 3 and the superior vena cava 5 on a map showing these structures, resulting in said second landmark. In another preferred embodiment, an ultrasound image of the right superior pulmonary vein 6 is taken, on which subsequently said first landmark is indicated on the junction between the left atrium 2 and the right superior pulmonary vein 6, after which this first landmark is directly projected on the junction between the right atrium 3 and the superior vena cava 5 on a map showing these structures, resulting in said second landmark. Said angiography may be a three-dimensional angiography which delivers a three-dimensional image which can be rotated for assisting in the indication of said first and/or second landmarks. Said angiography may also be performed by performing a film in a left anterior oblique (LAO) view 50°, which delivers a monoplane picture that is helpful in the indication of said first and/or second landmarks, for example by merging said monoplane picture with another of said images and/or maps. Said first and second landmarks may be a visually perceived and mentally determined location and/or may be a location that is captured on an image and/or map either manually, digitally and with or without the assistance of imaging or image processing tools. Said first and/or second landmarks may be in the form of a group of points, an aligned group of points, in the form of a continuous or non-continuous line, or in the form of another type of point clustering. Preferably, said first and/or second landmarks are selected as continuous or non-continuous lines, which are also referred to as first 17 and second landmark lines 20 in the current text.

Such construction of said second landmark is perfectly suitable for an accurate, easy and fast determination of at least one targeted location corresponding to a specific limited location 7. The perpendicular projection from the first landmark, indicated on the junction between the left atrium 2 and the right superior pulmonary vein 6 on one or more images and/or maps, onto the junction between the right atrium 3 and the superior vena cava 5 on one or more images and/or maps is a fast, accurate and reliable way for indicating a second landmark which comprises at least one specific limited location 7 of the heart where ablation is to be performed for an aimed treatment of sick sinus syndrome and other medical conditions characterized by abnormal sinus bradycardia.

In a preferred embodiment, the present invention provides a method according to the method of the invention, wherein before the step of directing energy from the ablation catheter towards tissue at a targeted location for ablation thereof, the method comprises the step of acquiring a first image and/or map that at least shows the junction between the heart's left atrium 2 and right superior pulmonary vein 6 and the step of acquiring a second image and/or map that at least shows the heart's right atrium 3 and superior vena cava 5, and subsequently the step of indicating a first landmark line 17 on the junction between the left atrium 2 and right superior pulmonary vein 6 on said first image and/or map, and the step of indicating a second landmark line 20, which second landmark line 20 comprises at least one targeted location corresponding to a specific limited location 7, by performing a perpendicular projection from said first landmark line 17 on said first image and/or map onto the junction between the right atrium 3 and the superior vena cava 5 on said second image and/or map.

Said images and/or maps may be two- or three-dimensional, preferably three-dimensional, and may be obtained by applying any known imaging and/or image processing tools as known in the state of the art. In an embodiment, images and/or maps are acquired by applying one or more CT scans. In other embodiments, three-dimensional image data are recorded by use of a X-ray CT scan and/or magnetic resonance tomography. In still other embodiments, images and/or maps are acquired by right pulmonary angiography or by taking ultrasound images. In a preferred embodiment, a CT scan image of the heart that at least shows the junction between the left atrium 2 and right superior pulmonary vein 6 is selected as said first image and a CT scan image of the heart that at least shows the right atrium 3 and superior vena cava 5 is selected as said second image. Said projection may be performed manually or automatically, preferably automatically. The step of indicating a first landmark line 17 and/or the step of indicating a second landmark line 20 may be performed by visually indicating a first 17 and/or second landmark line 20. With the term “visually indicating” it is meant that a resulting indication can be observed visually. For example, this may correspond to said first 17 and/or second landmark lines 20 that are digitally shown on said first and/or second images and/or maps, respectively. Using one or more advanced software programs, said first 17 and/or second landmark lines 20 may additionally or alternatively be indicated by pre-established image recognition, for example by pre-established image recognition of specific shapes of structures and/or of specific angulation between structures.

Such construction of said second landmark line 20 is perfectly suitable for an accurate, easy and fast determination of at least one targeted location corresponding to a specific limited location 7. The perpendicular projection from the first landmark line 17, indicated on the junction between the left atrium 2 and the right superior pulmonary vein 6 on said first image and/or map, onto the junction between the right atrium 3 and the superior vena cava 5 on said second image and/or map is a fast, accurate and reliable way for indicating a second landmark line 20 which comprises at least one specific limited location 7 of the heart where ablation is to be performed for an aimed treatment of sick sinus syndrome and other medical conditions characterized by abnormal sinus bradycardia.

In a preferred embodiment, the present invention provides a method according to the method of the invention, wherein said first and second images and/or maps are selected as first and second CT images and wherein between the step of indicating a first landmark line 17 on said first CT image and the step of indicating a second landmark line 20 on said second CT image, said first and second CT images are rotated in at least one direction with respect of each other in order to facilitate said perpendicular projection from said first landmark line 17 on said first CT image onto the junction between the right atrium 3 and the superior vena cava 5 on said second CT image for indicating the second landmark line 20.

Said rotation of said first and second CT images with respect of each other enables an accurate perpendicular projection from said first landmark line 17 on said first CT image onto the junction between the right atrium 3 and the superior vena cava 5 on said second CT image for indicating the second landmark line 20. Said rotation may be performed in one or more directions to establish an orientation of said first and second CT images with respect to each other which enables an accurate projection from said first landmark line 17 on said first CT image onto the junction between the right atrium 3 and the superior vena cava 5 on said second CT image. Said rotation may be performed manually or automatically and is preferably performed automatically.

In a preferred embodiment, the present invention provides a method according to the method of the invention, wherein said second landmark line 20 is indicated on said second image and/or map by establishing at least two spatially differentiated projections from the first landmark line 17 on said first image and/or map which are perpendicularly oriented with regard to the first landmark line 17, which projections are each ending on a point location on the junction between the right atrium 3 and the superior vena cava 5 on said second image and/or map, which point locations are subsequently connected to obtain said second landmark line 20.

Said at least two spatially differentiated projections may be present in a number from two projections to an approximately infinite number of projections. Said approach of establishing at least two spatially differentiated projections as described above is ideally suited to be automated.

In a preferred embodiment, the present invention provides a method according to the method of the invention, wherein said first 17 and/or second landmark lines 20, and more preferably said first 17 and second landmark lines 20, have a length of between 3 mm and 17 mm, more preferably of between 5 mm and 15 mm.

Such length is ideally suited for acquiring a second landmark line 20 which effectively comprises at least one specific limited location 7 of the heart where ablation is to be performed for an aimed treatment of sick sinus syndrome and other medical conditions characterized by abnormal sinus bradycardia.

In a preferred embodiment, the present invention provides a method according to the method of the invention, wherein after the step of indicating a second landmark line 20 on a second image and/or map and before the step of directing energy from the ablation catheter towards tissue at a targeted location for ablation thereof, mapping is performed of at least the heart's right atrium 3 and superior vena cava 5, after which a map resulting from the mapping is merged with an image comprising at least the heart's right atrium 3 and superior vena cava 5 together with the same heart's left atrium 2 and right superior pulmonary vein 6.

A resulting merged image is from a visual point of view very suitable for selecting at least one specific limited location 7 along said second landmark line 20 where ablation is to be performed for an aimed treatment of sick sinus syndrome and other medical conditions characterized by abnormal sinus bradycardia.

In a preferred embodiment, the present invention provides a method according to the method of the invention, wherein during and/or after ablation, preferably both during and after ablation, the evolution of heart parameters comprising heart rate and/or P-P interval are presented graphically. Such graphical presentation of heart parameters helps in performing an effective ablation according to the present invention.

In a preferred embodiment, the present invention provides a method according to the method of the invention, wherein ablation parameters, comprising an amount of energy to be applied for ablation and/or duration of applying energy for ablation, are based on pre- and per-procedural data. This approach helps in determining adequate ablation parameters in a quick and reliable way. Such pre- and per-procedural data may concern data of previous patients and/or animal data and procedure-related data. Such data may be anatomical data or may consist of functional data like time-P-P-interval curves. The ablation parameters could be indicative or automatically determined. In preferred embodiments, it is possible to select between an automatic and manual mode of determining the ablation parameters. In preferred embodiments, optimal places to start ablation and best consecutive ablation sites are based on pre-ablation data and procedural data. In preferred embodiments, it is automatically or manually determined when a pre-established endpoint of ablation is reached.

Another embodiment of the present invention concerns a system suitable for the ablation of tissue at the ablation target 7 at level of the junction between the right atrium 3 and the superior vena cava 5. This system comprises a flexible catheter, configured to be brought into contact with targeted tissue, at least one position sensing device in the catheter and an ablator, which applies a dosage of energy to the said targeted tissue. Besides, the system comprises circuitry for detecting electrical activity in the heart via at least one electrode on the catheter, a display, and a processor linked to the display and the circuitry, the processor operative for constructing electroanatomical maps of the heart. The components corresponding to this system are well-known and commonly available. To exemplify a system configured for applying ablation to cardiac structures, reference is made to U.S. Patent Application Publication No. 2013/0123773. For the system suitable for performing the ablation method of the present invention, it is of importance that the catheter can be navigated precisely and is able to focus energy deep within the targeted tissue.

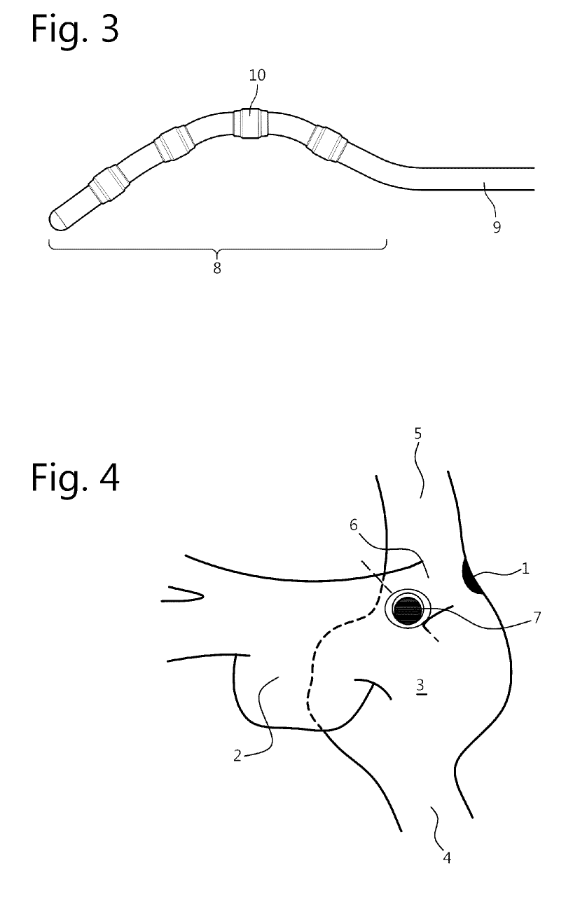

In a preferred embodiment of the present invention, the catheter, for use in the ablation system for performing the ablation method according to embodiments of the present invention, is designed in such a way that the catheter can optimally reach the ablation target 7. In FIG. 2, a diagram of a possible design of such catheter according to the preferred embodiment is shown. This catheter comprises a distal assembly 8 and a shaft 9. On the distal assembly 8, at least one electrode 10 is present. Illustratively, a multielectrode assembly, comprising four electrodes 10 at the distal assembly 8, is shown in FIG. 2. The angle between shaft 9 and distal assembly 8, as well as the angulation or curvature of the distal assembly 8 itself, can be modified for this catheter. These possibilities for angulation provide a considerable extent of flexibility to the distal assembly 8 of the catheter, facilitating the positioning of one or more electrodes 10 towards the ablation target 7. It should however be mentioned that this catheter could also be applied for ablative treatments at other locations. By preference, the angle between shaft 9 and distal assembly 8 and the angulation or curvature of the distal assembly 8 itself, can be modified using a steering mechanism. Such steering mechanism can preferably both control longitudinal motion (advance/retract) of the catheter and transverse motion (deflection/steering) of the distal assembly of the catheter. In FIG. 3, a three-dimensional representation of a possible design of said catheter according to the preferred embodiment is shown.

As the ablation method of the present invention is not restricted to a particular type of energy, the system suitable for performing this ablation method is not limited to deliver a particular energy type. According to a preferred embodiment of the invention, the energy for ablation applied by the above mentioned system can correspond to radiofrequency energy, microwave energy, cryogenic cooling or ultrasound energy. Alternatively to these types of ablation energy, applied by an ablation catheter, radiotherapy, e.g. proton therapy, could be used for targeting tissue at the specific limited location 7. Inclusion of the additional option of radiotherapy treatment would imply the extension of above mentioned system with a device for performing radiotherapy.

In a preferred embodiment, the present invention provides a system according to the system of the invention, whereby the catheter is provided with a means for irrigating the catheter. In other embodiments, the catheter may however be constructed without irrigation means.

In a preferred embodiment, the present invention provides a system according to the system of the invention, whereby the distal assembly 8 of the catheter comprises more than one electrode 10, more preferably 2 to 10 electrodes 10 and most preferably 3 to 8 electrodes 10. In a very preferred embodiment the distal assembly 8 comprises 5 electrodes 10.

In a preferred embodiment, the present invention provides a system according to the system of the invention, whereby the distal assembly 8 comprises at least one electrode 10 which is aligned with the shaft 9 of the catheter. In other words, the distal assembly 8 is aligned with the shaft 9 according to this embodiment. This alignment results in a shape of the catheter which is better adapted to reach the specific limited location 7 according to the present invention than a catheter with a distal assembly 8 being oriented perpendicularly to a shaft 9.

In embodiments, the catheter comprises a distal assembly 8 which is generally circular with a dimension of 5 to 15 mm. A plurality of electrodes 10, which can be any number of electrodes 10, yet preferably six, is dispersed on the generally circular portion of the distal assembly 8. The electrodes 10 are preferably ring electrodes. The most distal electrode 10 being approximately 1 to 5 mm from an atraumatic tip which is preferably a polyurethane plug at a distal tip of the distal assembly 8. Each electrode 10 is approximately 2 to 4 mm in length and is spaced from the next electrode 10 by approximately 3.5 to 5 mm. Each electrode 10 is made of a noble metal, preferably a mixture of platinum and iridium although other noble metals such as gold and palladium may also be used, and is connected to a plurality of wires, preferably lead wires. Each electrode 10 may be used for visualization, stimulation and ablation purposes. A thermocouple is preferably attached to each electrode 10 to provide an indication of the temperature at or near the tissue. Radiofrequency energy can be delivered either individually to one electrode 10, simultaneously to more than one electrode 10 or in a bi-polar mode between electrodes 10. The electrodes 10 are preferably irrigated, and may be irrigated through a plurality of apertures connected to an irrigation lumen.

In embodiments, the distal assembly 8 also comprises one or more sensors. In specific embodiments, the distal assembly comprises 3 sensors which may be three-axis magnetic location sensors or singles axis sensors. A distal sensor is located near the distal end of the most distal electrode 10. A middle sensor is located near the distal end of the electrode 10 located near an intermediate or middle electrode 10. A proximal sensor is a “floating sensor” located near the atraumatic tip. The catheter alternatively contains a contraction wire that is used to vary the expansion and contraction of the general circular assembly, which assembly is hereinafter also called “loop”, to varying sizes. Such a contractible catheter could be made in two size ranges: one varying from between approximately 19 mm in diameter at the largest down to approximately 10 mm at its smallest fully contracted state; and a second smaller diameter catheter varying between approximately 14 mm in diameter at its largest down to approximately 6 mm at its smallest fully contracted state. If a contraction wire is not used the distal assembly 8 should be approximately 8 to 12 mm and preferably around 10 mm in diameter when unconstrained. Such distal assembly 8 is preferably aligned with the shaft 9 of the catheter. The distal assembly 8 may however also be designed to define an arc oriented obliquely relative to the axis and having a center of curvature on the axis. The term “oblique” in the present context means that the plane in space that best fits the arc is angled relative to the longitudinal axis of shaft 9. The angle between the plane and the axis is greater than 45 degrees. The arc subtends 180 degrees forming a semicircle which can then be contracted into a smaller circular shape. The angle of the subtended arc may vary from 90 degrees to 360 degrees, but in a preferable embodiment is 180 degrees.

In embodiments, the loop includes a base which is connected to the distal end of the shaft 9 and a tip. The loop features are centered, generally cylindrical form such that the tip protrudes axially in a distal direction relative to the base. Preferably, the axis of the base and shaft 9 is centered along the diameter of the unconstrained loop, however, it may also be centered along the diameter of the constrained loop. The pitch of the distal assembly 8 is fixed along the length of the loop and is approximately 5 to 20 degrees.

The shape of the distal assembly 8 arises by incorporating a structure made from a shape memory material such as nitinol which has been pre-formed to assume the desired shape when unconstrained at body temperature. The distal assembly 8 is sufficiently flexible to permit the loop to straighten during insertion through a sheath and then resume the arcuate form when unconstrained.

In embodiments, the shaft 9 of the catheter is attached to a control handle which has a narrower portion at the proximal end of the shaft 9. Control handle may alternatively include two independent mechanisms for controlling the expansion/contraction of the loop through a contraction wire and the deflection of the distal tip assembly using a puller wire.

In embodiments, the catheter may also incorporate a guidewire to ensure placement of the distal assembly 8 at the proper location or it may incorporate a soft distal tip section parallel to the longitudinal axis of the shaft 9 and base that would be used to guide the distal assembly 8 into a proper location.

In embodiments, abovementioned control handle is a generally cylindrical tubular structure but can also take other shapes and configurations that provide the user of the system with the ability to manipulate the catheter while providing an interior cavity for passage of components. Control handle comprising a narrower portion is made of an injection molded polymer such as polyethylene, polycarbonate or ABS or other similar material. A connector is preferably inserted into the proximal end of control handle and provides an electrical connection to a mating connector and cable assembly that is connected to a radiofrequency generator. Connector is secured through the use of epoxy or other similar means.

A lead wire assembly preferably comprising a Teflon sheath and six pairs of lead wires is housed therein, one pair for each electrode 10 and associated thermocouple. The proximal end of each lead wire is electrically and mechanically connected to the connector through the use of solder or other means. An irrigation luer hub is a fitting capable of being attached to mating connector from an irrigation source such as an irrigation pump. An irrigation luer hub is attached to irrigation side arm using polyamide to form a seal against fluid intrusion. Irrigation fluid is then conveyed from the irrigation hub through the irrigation lumen. Irrigation lumen passes through the lumen in a side arm through the wall of the control handle through the shaft 9 and then into an irrigation lumen in the base of the multi-lumen tube for approximately 3 mm into the distal assembly 8 in order to convey irrigation fluid to each electrode 10 which has a plurality of holes apertures 519 therethrough. The catheter may also be constructed without irrigation.

In embodiments, said control handle has a portion of a smaller diameter which is adapted to receive the proximal end of the catheter which is preferably comprised of a strain relief element and shaft 9 through which preferably a lead wire assembly and irrigation lumen pass. Strain relief elements in a preferred embodiment are two shrink sleeves made of polyolefin or similar material which are heated to shrink over the shaft 9. Polyurethane is then used to attach the strain relief elements into the handle portion.

In embodiments, the working length of the catheter is approximately 80 to 100 cm from the distal end of a strain relief element to the distal tip of the distal assembly 8. The working length may however vary depending on the application. In embodiments, the distal assembly 8 comprises a multi-lumen tube which has a plurality of electrodes 10 mounted thereon. In a preferred embodiment, four electrodes are used. The maximum diameter of the generally circular distal assembly 8 is approximately 8-12 mm, preferably around 10 mm when un-constricted. The electrodes 10 are preferably ring electrodes and preferably have a maximum outer diameter of 2 mm at the middle and a minimum outer diameter of 1.7 mm at the narrower ends. The electrodes 10 may be made of any material but are preferably made of 90% platinum and 10% iridium but could be comprised of a combination of these and/or other suitable noble metals such as gold and palladium. A multi-lumen tube with a base is made of a material that is more flexible than the material in the shaft 9, and is preferably 35D PEBAX with no wire braid, although other materials and durometers may be used depending on the desired stiffness of the distal assembly 8. Shaft 9 is preferably made of pellethane, polyurethane or PEBAX and contains an internal stiffener which is an inner tube made of nylon or polyimide or similar material.

In embodiments, the catheter comprises at least one pair of lead wires, with each pair of lead wires being welded to a respective electrode 10 to provide a robust connection. A polyurethane coating is placed over each end of each electrode 10 in order to seal against a fluid intrusion and to provide an atraumatic transition between the electrodes 10 and the multi-lumen tube of distal assembly 8. In embodiments, the catheter comprises an atraumatic tip dome which is preferably a polyurethane dome with a shaft 9 that extends into the end of the irrigation lumen at the end of a multi-lumen tube. A nitinol wire/shape memory support member extends from at or near the distal end of the multi-lumen tube into the shaft 9 for approximately 25 millimeters into the shaft 9. This provides stability to the distal assembly 8. Nitinol wire is preferably square in cross-section, and preferably 0.0075 inch by 0.0075 inch, but could be square, circular or rectangular in cross-section with a width or diameter between 0.006 inch and 0.010 inch. The nitinol wire is pre-formed to take a generally circular shape having a diameter of approximately 10 mm and a height of approximately 5 to 11 mm preferably approximately 7 mm when it is in not constrained within a sheath. The nitinol wire will impart this circular shape on the other components of the distal assembly 8.

In embodiments, the catheter comprises a multi-lumen tube which also contains an irrigation lumen and a lead wire lumen housing a lead wire assembly which comprises pairs of lead wires. A lumen houses the nitinol wire. A lumen in the multi-lumen tube may be unused. Such lumen could however be used for a contraction wire, wiring for additional thermocouples or other sensors that are desired in the distal assembly 8.

In embodiments, the shaft 9 comprises a stiffener which provides added stiffness to the shaft 9 and is comprised of a material such as polyimide or nylon, preferably polyimide having a thickness of approximately 0.002 thousandths of an inch. The stiffener runs substantially along the entire length of the shaft 9. Polyurethane is preferably used to bond the shaft 9 to the base of the multi-lumen tube. This preferred polyurethane bond prevents fluids from entering at the junction of these two elements. Other methods of bonding such as heat sealing or other glues may be used.

In embodiments, a fluoro-opaque marker may additionally be placed at or near the distal end of the distal assembly 8 to aid visualization under fluoroscopy. Such a fluoro-opaque marker can be a ring shaped structure made from a noble metal such as a combination of platinum and iridium of a similar composition to an electrode 10, preferably to a circular electrode, however such a marker band may be narrower in width and would not contain apertures for irrigation fluid.

In embodiments, the catheter is used with a sheath, preferably a steerable sheath which facilitates the placement of the catheter in the proper place in the anatomy for the desired ablation. Once the distal end of the catheter exits the sheath a nitinol wire/support member will cause the distal assembly 8 to take the pre-configured generally circular shape.

In a preferred embodiment, the present invention provides a system according to the system of the invention, whereby the system is equipped and configured to be able to graphically present the evolution of heart parameters, comprising heart rate and/or P-P interval, during and/or after ablation, preferably both during and after ablation. Such graphical presentation of heart parameters helps in performing an effective ablation according to the present invention.

In a preferred embodiment, the present invention provides a system according to the system of the invention, wherein the system is equipped and configured to base ablation parameters, comprising an amount of energy to be applied for ablation and/or duration of applying energy for ablation, on pre- and per-procedural data. This approach helps in determining adequate ablation parameters in a quick and reliable way. Such pre- and per-procedural data may concern data of previous patients and/or animal data and procedure-related data. Such data may be anatomical data or may consist of functional data like time-P-P-interval curves. The ablation parameters could be indicative or automatically determined. In preferred embodiments, it is possible to select between an automatic and manual mode of determining the ablation parameters. In preferred embodiments, optimal places to start ablation and best consecutive ablation sites are based on pre-ablation data and procedural data. In preferred embodiments, it is automatically or manually determined when a pre-established endpoint of ablation is reached.

Another embodiment of the invention concerns a manual for the ablation of tissue at the ablation target 7 at level of the junction between the right atrium 3 and the superior vena cava 5, in accordance with the previous embodiments of the present invention. This specific manual contains explanatory text as well as illustrative drawings, which can be used by a practiced physician as guidelines to perform treatments according to the present invention.

In a specifically preferred embodiment, ablation of the specific limited location 7 is performed by the system of the invention and according to the method of the invention.

Another aspect of the present invention concerns a catheter according to the embodiments described above for the system. Such an individual catheter could be used for various cardiac procedures, and is especially suitable to perform the method of ablation according to the present invention.

### EXAMPLES

### Example 1

P-P interval (PPI) shortening as a function of time (t) during two applications of radiofrequency ablation at the specific limited location 7, according to embodiments of the present invention, is shown in FIG. 7. Each application of radiofrequency ablation (RFA) is executed for 60 seconds. Due to the first application of RFA the P-P interval drops from A to B. After the first application of RFA, the P-P interval increases again to level C. As a results of the second application of RFA, the P-P interval drops from C to D. Afterwards, the P-P interval rises again to level E, which was below the P-P level targeted for the ablation treatment.

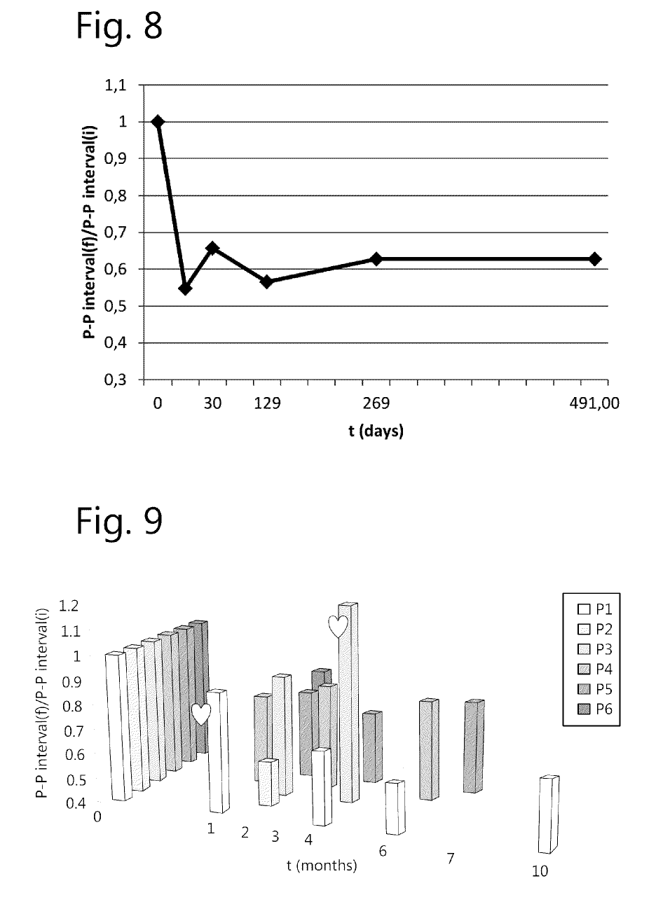

FIG. 8 is a graph showing the residual amount of the P-P interval value retained during follow up after ablation treatment at the specific limited location 7 according to an embodiment of the present invention. The results correspond to P-P interval values of a patient which was treated at the specific limited location 7 by a left atrial approach. The follow up was performed during more than one year. The time (t) after the ablation treatment is expressed in days. The P-P intervals were measured using an ECG registration device. The results are shown as a ratio of the P-P interval remained during follow up (P-P interval(f)) relative to the P-P interval prior to ablation (P-P interval(i)). From the graph it is obvious that the P-P interval decreased in time.

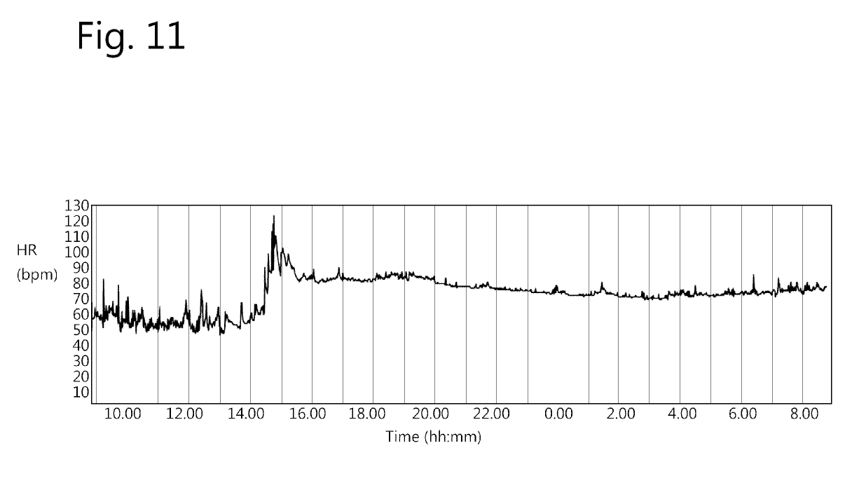

The results shown in FIG. 7 and FIG. 8 are related to ablation treatment of a 16 years-old woman having recurring prolonged syncopes since 5 years and without any structural heart disease had a sinus bradycardia of 43 beats per minute (bpm) at rest. She developed a pause of 17 s during Tilt test. After 2 applications of RFA (60′ s, 25 Watts, 3 actives electrodes) with a nMARQ™ catheter at the specific limited location 7 described in this text, her P-P interval shortened from 1089 ms to 680 ms (FIG. 7). The periprocedural HR modifications were tracked by an non invasive HR monitoring (FIG. 11). More than 2 years after procedure the patient remains completely asymptomatic. The P-P interval shortening was maintained after 491 days, as can be seen in FIG. 8.

### Example 2

FIG. 9 is a graph showing the residual amount of the P-P interval value during follow up after ablation treatment at a specific limited location 7 according to an embodiment of the present invention. The ablation treatment was performed by a right atrial approach. The results are shown as a ratio of the P-P interval remained during follow up (P-P interval(f)) relative to the P-P interval prior to ablation (P-P interval(i)). Six patients (P1 to P6) were monitored and the follow up was performed during multiple months. The P-P intervals were measured using a regular ECG registration device. Generally, the P-P interval shortened during follow up. The hearts indicate a higher vagal tonus during the ECG registration in two patients at a particular moment of follow up.

### Example 3

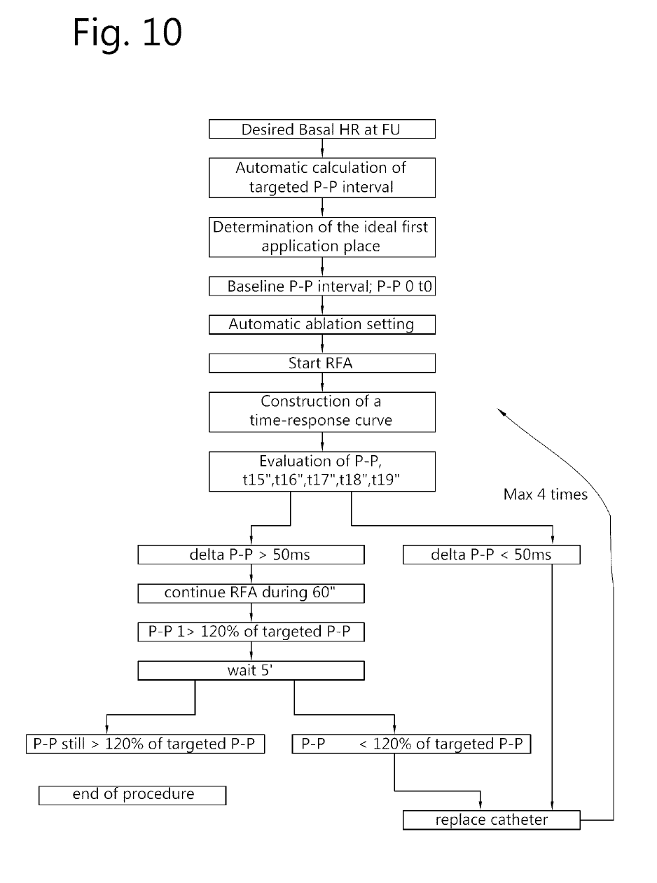

FIG. 10 is a schematical representation of an algorithm for ablation according to embodiments of the present invention.

Based on the concept of ablation on the specific limited location 7 according to the method of the present invention, on the targeted location 7, on biophysical available knowledge and on unpolished in vivo data of time-P-P interval curves during ablation, an algorithm is proposed to identify the preferred initial ablation site, to define ablation parameters, to define active ablation electrodes, to define the moment and the location of catheter repositioning, to define the amount of ablation lesions and to define the procedural endpoints.

Several parameters are of importance and are discussed in the following section:

### 1. Maximal Heart Rate (HR) Reached During a Pharmacological Screening Test:

A value must be encoded in the system. The minimal value of the P-P interval observed during this test gives an indication of the potential value of this therapy for an individual patient and will help him to perform an appropriate patient selection.

### 2. Desired Persistent Post Procedural Basal Heart Rate (HR):

This target heart rate (HR) must be encoded in the system. This value is defined as the heart rate desired after ablation treatment without, or with minimal autonomous nervous stimulation and without medications affecting the heart rate directly or indirectly. The system provides automatically the mean P-P interval corresponding to the value encoded. The theoretical maximal value of this parameter is patient related and depends especially from the maximal HR observed during the pharmacological screening test. Additional patient characteristics are of importance to determine this parameter. The system proposes a targeted P-P threshold who needs to be confirmed by an operator.

### 3. Type of Catheter Used:

This information must be provided. Catheter design and especially the number of ablating electrodes 10 is of importance for lesion creation within the target

### 4. Anatomical Characteristics:

The distance and the tissues located between the active electrode(s) 10 and the targeted location 7 are of importance and will limit the applicability of the method of ablation according to the present invention. Ideally, the target should be visualized. This is not possible yet with the techniques used in clinical practice during ablation treatments of cardiac structures. Those techniques, being endocardial electroanatomical mapping, CT scanning, angiography, provide a visualization of the endocardial limits of the structures surrounding the targeted location 7. This information is sufficient to apply this technique in routine clinical practice. Additional techniques able to visualize the wall thickness of the surrounding structures are theoretically of a bigger value and can be incorporated into the system as well.

### 5. Location of the Target:

Based on the imaging data collected, and on the interactions between the structures visualized, a theoretical 3-D location of the targeted location 7 will be automatically provided by the system. Targeted location and targeted volume will vary between patients. One of the ways to represent the targeted location is a core surrounded by concentric lines. For the purpose of simplicity, those lines will be circular or ellipsoidal.

### 6. Patient Selection:

The system will integrate information of steps 1, 4, 5 and will provides the physician important information on patient eligibility.

### 7. Evaluation of the P-P Interval During Ablation:

The P-P interval will be evaluated beat to beat. Variations of this interval beyond limits to be specified will be rejected by the system and not taken into account for the construction of the time- response curves. The values beyond this ‘confidence interval’ will also not be taken into account in the algorithm. The confidence interval is proposed by the system and can be adapted by the physician within certain limits.

### 8. Biological Effect Assessment:

The time interval and the importance of the biological response used by the system to assess biological efficacy during individual applications of radiofrequency ablation (RFA) are proposed by the system and can be adapted by the physician in certain limits.

Based on observed in vivo data a P-P interval shortening of 50 ms or more should be observed after 20 s of RFA or even better after 15 s of RFA. Those values can be adapted.

### 9. The First Application of RFA:

Based on the above mentioned information (steps 1, 2, 3, 4 and 5) an initial ablation place and an initial ablation setting will be proposed to the physician by the system but could be overruled in certain limits.

### 10. During Individual Ablation Lesions:

The system will assess quality of catheter contact and lesion formation based on established techniques. Endocardial sites already ablated will be mentioned by regular techniques. The system will also track P-P interval changes and will construct beat to beat time-P-P interval shortening curves.

### 11. Catheter Replacement:

Based on classical information during ablation, catheter contact and endocardial lesion creation will be assessed. Beside this classical information during ablation, the biological effect of each ablation will be tracked. Based on the location of the targeted location 7, on biophysical considerations and on in vivo data, the P-P interval shortening is evaluated after and during a strategic moment during each new application of RFA. The period initially proposed by the system to assess P-P interval shortening is between 15 s and 20′ s. This period can be adapted by the physician within certain limits. The catheter should be repositioned if no sufficient effect is observed. For example a shortening of more than 50 ms after 15 s to 20 s after the start of RFA could be used as minimal value to establish if the catheter needs to be repositioned or not. Once the biological efficacy of the ablation has been confirmed, RFA must be continued to reach a consolidated lesion within the targeted location 7. RFA could be interrupted if an excessive response is observed at a certain place or if the information collected during ablation suggests that the catheter is rather located in front of nervous extensions rather than in front of neural bodies. In order to avoid the occurrence of re-entrant arrhythmias, the system will also help the operator to make continuous lesions when performing more than one ablation.

### 12. Waiting Periods After Individual Ablations:

After each individual ablation, the P-P interval is compared with the desired persistent post procedural basal heart rate. If the observed P-P interval at that time exceeds a certain percentage, for example, 120%, of the P-P interval corresponding to the desired persistent post procedural basal heart rate, a new ablation is started immediately without any waiting time. On the contrary, if the observed P-P interval exceeds this predefined limit, a waiting period is started (for example 10 s). If the observed P-P interval drops under this level during the waiting time, a new ablation should be performed. The new ablation should be started at the moment a plateau phase has been reached during a few minutes during the waiting period. In order to approach the desired P-P interval as much as possible to really tailor the approach to patient's needs, ablation setting specific for this potential last lesion are proposed by the system to the operator and have to be validated. This is of important value for a targeted approach.

### 13. Patient Follow-Up and Continuous Education:

Both to perform research as to track the vagolysis effect during follow-up (FU) a specific pre and post procedural program and/ or device has an important additional value. Each physician performing this technique should have such a device containing both the important clinical and procedural data of the patients undergoing this procedure. The biological effect in function of time should be demonstrated to the physician by a figure for each patient, for which, for example, reference can be made to Examples 2 and 3. A collection of those data within a worldwide database will be available to promote research and optimize patient care.

### Example 4

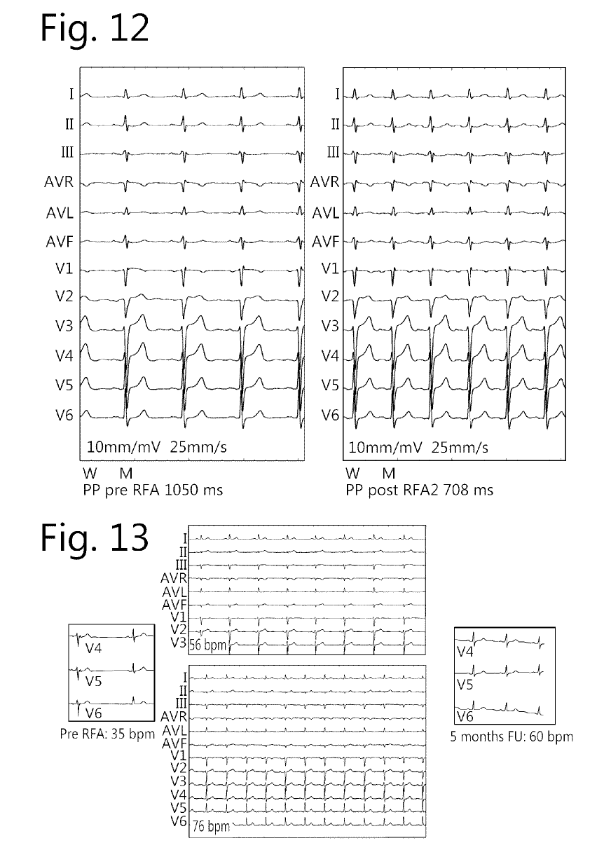

A 72 years old gentleman with a brady/tachy syndrome and frequent recurring syncopes underwent first a RFA at the specific limited location 7 described in this text and then a pulmonary vein isolation during a single procedure 8 months ago. After a single application of RFA (60′ s, 25 Watts, 3 actives electrodes) with a nMARQ™ catheter from the right atrium 3, his P-P interval decreased from 1050 to 852 ms. After a second application of RFA on the opposite site of the left atrium 2 with the same ablation settings, the P-P interval further shortened till 708ms. (FIG. 12). Eight months later, the patient is completely asymptomatic and the ‘resetted’ P-P interval shortening remained unchanged.

### Example 5

A 47 years-old gentleman with recurring syncopes underwent an ablation at the same site. We performed an electro-anatomical map of the right atrium 3 and the caval veins. The target location was identified after having performed a merge with a previous CT.

With this approach, the procedure is limited to the right side of the heart. After 3 applications of RFA with the same settings as for Examples 1 and 4, his basal HR accelerated from 56 to 76 bpm (FIG. 13). This biological change was maintained at 5 months follow up (FU). 363 days later, the patient is still completely asymptomatic.

### Example 6

In a 65 years man old, ablation at the specific limited location 7 was performed successfully by combining the electro-anatomical map of the right atrium 3 and of the caval veins with a contrast imaging of the right superior pulmonary vein 6. The venous return phase was filmed after a selective contrast injection in the right superior pulmonary vein 6. With this approach, a pre-procedural CT scan is not mandatory anymore. The patient had an uneventful follow-up.

### Example 7

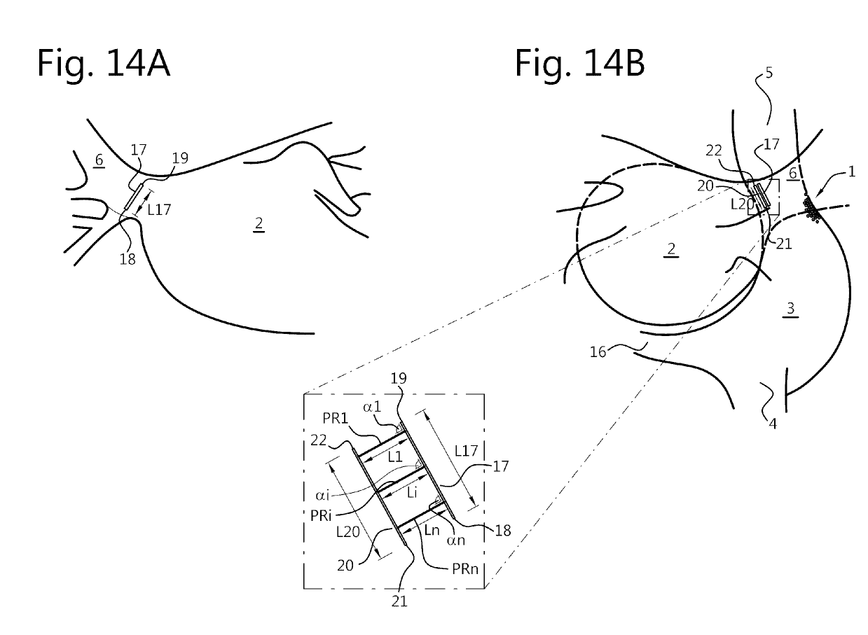

FIG. 14A-B are diagrams showing (A) a left atrium 2 and pulmonary veins of a heart in an anterior posterior view and (B) a left atrium 2, right atrium 3, pulmonary veins and caval veins of a heart in posteroanterior view, with indication of landmark lines 17, 20 for ablation, in accordance with an embodiment of the present invention. In FIG. 14A, a first landmark line 17 starting at a starting point 18 and ending at an end point 19 and having a length L17 of between 5 mm and 15 mm, is indicated at the junction between a left atrium 2 and a right superior pulmonary vein 6 of a heart. In FIG. 14B, it is shown that a second landmark line 20 starting at a starting point 21 and ending at an end point 22 and having a length L20 of between 5 mm and 15 mm, is indicated at the junction between a superior vena cava 5 and a right atrium 3 of said heart, but rather on the side of the superior vena cava 5, by performing a perpendicular projection of said first landmark line 17 onto said junction between a superior vena cava 5 and a right atrium 3 of said heart. Said perpendicular projection is preferably performed in an automated manner. A magnified part of FIG. 14B shows an approach according to which said perpendicular projection may be performed. Along said first landmark line 17, a number (1 to n) of projections PR1, PRi, PRn are made according to angles α1, αi, an which are oriented perpendicularly with regard to said first landmark line 17. Determination of shortest distances L1, Li, Ln of said projections PR1, PRi, PRn ending on the junction between the left atrium 2 and the right superior pulmonary vein 6 establishes a set of points which after connection result in said second landmark line 20. To enable said perpendicular projection, a first image that at least shows the junction between the heart's left atrium 2 and right superior pulmonary vein 6 and a second image that at least shows the heart's right atrium 3 and superior vena cava 5 are rotated with respect to each other in one or more directions to establish an orientation of said first and second images with respect to each other which enables an accurate projection from a first landmark line 17 at the junction between the left atrium 2 and the right superior pulmonary vein 6 on said first image onto the junction between the right atrium 3 and the superior vena cava 5 on said second image. Said rotation may be performed manually or automatically and is preferably performed automatically. When no three-dimensional image is available for the heart's left cavities, a left anterior oblique (LAO) view 50° is preferentially used.

### Example 8

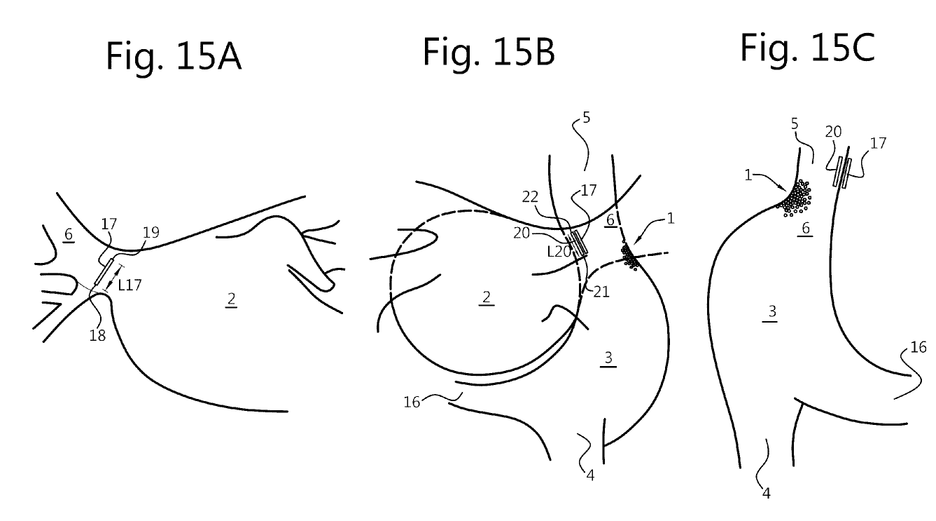

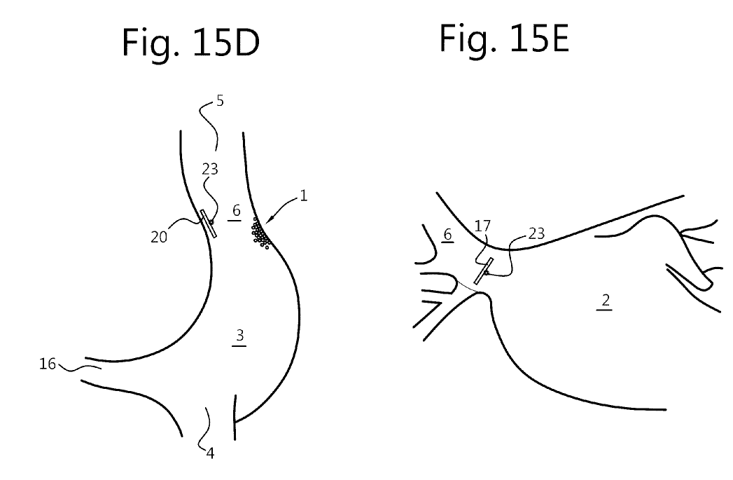

FIG. 15A-E show diagrams related to different steps intended for indicating landmark lines 17, 20 for ablation on a heart and for performing ablation at level of one of said lines, in accordance with an embodiment of the present invention. In a first step, shown in FIG. 15A, a first landmark line 17, preferably having a length L17 of between 5 mm and 15 mm, is indicated on a CT image on a junction between the left atrium 2 and a right superior pulmonary vein 6 of a heart. FIG. 15A shows an image in an anterior posterior view as obtained by a CT scan of said heart. In a second step, shown in a posteroanterior view of a CT image of said heart in FIG. 15B, a second landmark line 20, preferably having a length L20 of between 5 mm and 15 mm, is indicated at a junction between a superior vena cava 5 and a right atrium 3 of said heart by performing a perpendicular projection of said first landmark line 17 onto said junction between a superior vena cava 5 and a right atrium 3 of said heart. The indication of said second landmark line 20 may be performed as described in EXAMPLE 7. Afterwards, in a third step, right cardial structures, among which the right atrium 3, superior vena cava 5, inferior vena cava 4 and coronary sinus 16, are mapped, after which the resulting map is merged with the original CT image. The thus resulting image of heart structures is shown according to a left anterior oblique view in FIG. 15 C. The view shown in FIG. 15C is very suitable for selecting a point location 23 on the second landmark line 20 where ablation is to be performed. In a fourth step, ablation is performed at such point location 23, as shown in a posteroanterior view according to FIG. 15D. Finally, in a fifth step, it is illustratively shown in an anterior posterior view that, said point location 23 is also located along said first landmark line 17.

What is claimed is:

1. Method of ablation designed for the treatment of sick sinus syndrome and other medical conditions characterized by abnormal sinus bradycardia, comprising the steps of: inserting an ablation catheter into a heart of a living subject, and directing energy from the ablation catheter towards tissue at a targeted location for ablation thereof, wherein the targeted location corresponds to a specific limited location at level of the junction between the right atrium and the superior vena cava.

2. The method of claim 1, wherein the specific limited location is targeted from the endocardial side of the right atrium.

3. The method of claim 2, wherein the specific limited location corresponds to a definite small region of a few millimeters at the posterior side of the junction between the superior vena cava and the right atrium and opposed to the junction of the right superior pulmonary vein with the left atrium.

4. The method of claim 3, wherein the specific limited location ranges from 5 to 10 millimeters in diameter.

5. The method of claim 4, wherein the anterior right ganglionated plexi are targeted for ablation at the specific limited location.

6. The method of claim 5, wherein sinus node acceleration is obtained during the ablation treatment.

7. The method of claim 6, wherein energy applied for ablation and the surface of the ablation area are determined by evaluating sinus rhythm acceleration.

8. The method of claim 7, wherein the energy applied for ablation is automatically regulated according to the extent in which a predefined amount of sinus node acceleration is reached.

9. The method of claim 1, wherein the energy used for ablation comprises radiofrequency energy, laser energy, microwave energy, cryogenic cooling or ultrasound energy.

10. The method of claim 1, wherein the specific limited location corresponds to a definite small region of a few millimeters at the posterior side of the junction between the superior vena cava and the right atrium and opposed to the junction of the right superior pulmonary vein with the left atrium, at level of the inferior and mid parts of the right superior pulmonary vein.

11. The method of claim 3, wherein at least identification of the specific limited location, imaging of the right superior pulmonary vein, ablation at the specific limited location, screening of the patients prior to ablation, and/or follow up of patients after ablation, is regulated by an algorithm.

12. The method of claim 1, wherein before the step of directing energy from the ablation catheter towards tissue at a targeted location for ablation thereof, the method comprises the step of acquiring an assembly of one or more images and/or maps which as a whole at least show the junction between the heart's left atrium and right superior pulmonary vein, as well as the heart's right atrium and superior vena cava, and subsequently the step of indicating a first landmark on the junction between the left atrium and right superior pulmonary vein on one or more images and/or maps, and the step of indicating a second landmark, which second landmark comprises at least one targeted location corresponding to a specific limited location, by performing a perpendicular projection from said first landmark on the junction between the right atrium and the superior vena cava on one or more images and/or maps.

13. The method of claim 1, wherein before the step of directing energy from the ablation catheter towards tissue at a targeted location for ablation thereof, the method comprises the step of acquiring a first image and/or map that at least shows the junction between the heart's left atrium and right superior pulmonary vein and the step of acquiring a second image and/or map that at least shows the heart's right atrium and superior vena cava, and subsequently the step of indicating a first landmark line on the junction between the left atrium and right superior pulmonary vein on said first image and/or map, and the step of indicating a second landmark line, which second landmark line comprises at least one targeted location corresponding to a specific limited location, by performing a perpendicular projection from said first landmark line on said first image and/or map onto the junction between the right atrium and the superior vena cava on said second image and/or map.

14. The method of claim 13, wherein said first and second images and/or maps are selected as first and second CT images and in that between the step of indicating a first landmark line on said first CT image and the step of indicating a second landmark line on said second CT image, said first and second CT images are rotated in at least one direction with respect of each other in order to facilitate said perpendicular projection from said first landmark line on said first CT image onto the junction between the right atrium and the superior vena cava on said second CT image for indicating the second landmark line.

15. The method of claim 13, wherein said second landmark line is indicated on said second image and/or map by establishing at least two spatially differentiated projections from the first landmark line on said first image and/or map which are perpendicularly oriented with regard to the first landmark line, which projections are each ending on a point location on the junction between the right atrium and the superior vena cava on said second image and/or map, which point locations are subsequently connected to obtain said second landmark line.

16. The method of claim 13, wherein said first and/or second landmark lines have a length of between 5 mm and 15 mm.

17. The method of claim 13, wherein after the step of indicating a second landmark line on a second image and/or map and before the step of directing energy from the ablation catheter towards tissue at a targeted location for ablation thereof, mapping is performed of at least the heart's right atrium and superior vena cava, after which a map resulting from the mapping is merged with an image comprising at least the heart's right atrium and superior vena cava together with the same heart's left atrium and right superior pulmonary vein.

18. The method of claim 1, wherein during and/or after ablation, the evolution of heart parameters comprising heart rate and/or P-P interval are presented graphically.

19. The method of claim 1, wherein ablation parameters, comprising an amount of energy to be applied for ablation and/or duration of applying energy for ablation, are based on pre- and per-procedural data.

20. System for the ablation of tissue at the targeted location at level of the junction between the right atrium and the superior vena cava, comprising:
a flexible catheter, configured to be brought into contact with targeted tissue; at least one position sensing device in the catheter; an ablator, which applies a dosage of energy to the said targeted tissue; circuitry for detecting electrical activity in the heart via at least one electrode on the catheter; a display; and a processor linked to the display and the circuitry, the processor operative for constructing electroanatomical maps of the heart.

21. The system of claim 20, wherein the catheter comprises a shaft and a distal assembly, the distal assembly comprising at least one electrode, for which the angle between shaft and distal assembly, as well as the angulation or curvature of the distal assembly itself, can be modified.

22. The system of claim 20, wherein the energy applied for ablation comprises radiofrequency energy, laser energy, microwave energy, cryogenic cooling or ultrasound energy.

23. Manual for the ablation of tissue at the targeted location at level of the junction between the right atrium and the superior vena cava.
